(19) United States
(12) Patent Application Publication
(10) Pub. No.: US 2012/0288544 A1
(43) Pub. Date: Nov. 15, 2012

(54) NOVEL RETIGABINE COMPOSITION

(75) Inventors: Matthew Burke, Research Triangle Park, NC (US); Charles Harding Rhodes, Research Triangle Park, NC (US)

(73) Assignee: GLAXO GROUP LIMITED, Greenford, Middlesex (GB)

(21) Appl. No.: 13/574,077

(22) PCT Filed: Jan. 18, 2011
(86) PCT No.: PCT/EP2011/050633

§ 371 Date: Jul. 19, 2012

(30) Foreign Application Priority Data
Jan. 20, 2010 (US) 61296652
Jan. 21, 2010 (US) 61297038

(51) Int. Cl. A61K 9/00; A61K 31/24

(52) U.S. Cl. 424/400; 514/535

(57) ABSTRACT

The present invention relates to an oral dosage form comprising N-(2-amino-4-(fluorobenzylamino)-phenyl)carbamic acid ethyl ester (retigabine) or a pharmaceutically acceptable salt or solvate thereof, to a process for preparing such a dosage form and to the use of such a dosage form in medicine.

The present invention relates to an oral dosage form comprising N-(2-amino-4-(fluorobenzylamino-phenyl)carbamic acid ethyl ester (retigabine) or a pharmaceutically acceptable salt or solvate thereof, to a process for preparing such a dosage form and to the use of such a dosage form in medicine.

Retigabine was disclosed in U.S. Pat. No. 5,384,330, and polymorphs of retigabine including polymorph A were disclosed in U.S. Pat. No. 6,538,151 and their process for manufacture in U.S. Pat. No. 5,914,425.

Controlling the rate of release of an active agent from an oral dosage form has received considerable attention and many different devices have been developed for such a purpose. U.S. Pat. No. 5,004,614 describes a tablet core provided with an outer coating that is substantially impermeable to environmental fluid. The said outer coating may be prepared from materials that are either insoluble or soluble in the environmental fluids. Where a soluble material is used, the coating is of sufficient thickness that the core is not exposed to environmental fluid before the desired duration of the controlled release of the active agent has passed. Through this impermeable outer coating, one or more opening(s) has been created, so as to provide environmental fluids with an access route to the core. Therefore, upon ingestion of the coated tablet, gastro-intestinal fluid can enter the openings(s) and contact or penetrate the core, to release the active agent. The result is that the active agent is released in a controlled manner out of the opening(s) only. The preferred geometry is such that there is a circular hole on the top and bottom face of the coated tablet. The opening(s) in question have an area from about 10 to 60 percent of the face area of the coated tablet. The rate of drug release is found to be directly related to the diameter of the opening(s) and to the solubility of the matrix core and active agent, allowing the possibility for a variety of drug release profiles be it zero or first order release.

The substantially impermeable coatings of U.S. Pat. No. 5,004,614 are not suitable for the controlled release of all active agents, especially pharmaceutically active weak bases or pharmaceutically acceptable salts and solvates thereof. Such active agents exhibit a marked pH dependent solubility, i.e. they are more soluble at around pH 2, associated with regions found in the stomach, compared to their solubility in the generally neutral conditions of the small intestine, around pH 7.

WO2005/013935 discloses an oral dosage form comprising a first composition and a second composition, each composition comprising a pharmaceutically acceptable weak base, especially (5-[4-[2-(N-methyl-N-(2-pyridyl)amino)ethoxy]benzyl]thoazolidine-2,4-dione (herein after “Compound A”) or a pharmaceutically acceptable salt or solvate thereof, (“the drug”) and a pharmaceutically acceptable carrier thereof, wherein the first and second compositions are arranged to release drug at difference release rates on administration such that the rate of release of the drug from the dosage form is substantially independent of pH.

U.S. application Ser. No. 12/505,409 and International Patent Application No. PCT/US2009/051052 (WO2010/009433) disclose modified release pharmaceutical formulations including about 30-70% retigabine or a pharmaceutically acceptable salt, solvate or hydrate thereof, about 5-30% of a drug delivery matrix including hydroxpropylmethylcellulose (HPMC), about 1.0-10% of an anionic surfactant, and an enteric polymer. Formulations including about 30-70% retigabine, or a pharmaceutically acceptable salt, solvate or hydrate thereof, about 5-30% drug delivery matrix, and an agent for retarding release in the gastric environment are also disclosed.

Retigabine is a neuronal potassium channel opener currently in late-state development as an adjunctive treatment for adult patients with partial-onset seizures. In Phase III epilepsy trials, retigabine reduced seizure rates compared to patients taking placebo. Retigabine may also be useful fro treating a variety of disorders characterised by nervous system hyperexcitability and/or smooth muscle hyperexcitability including seizure disorders such as epilepsy, neuopathic pain, inflammation, overactive bladder, urinary incontinence, functional bowel disorders, ulcerative conditions of the intestinal tract, hyperactive gastric motility, asthma, hypertension migraine and eating disorders. Generally pharmaceutical compositions containing retigabine may be useful as antidystonics, effectively reducing muscle tonicity and spasms. Additionally retigabine may also be useful as a neuroprotective agent, for example, under conditions of reduced cerebral blood flow, such as during a stroke and other ischemia-related events, and for the treatment of vascular diseases affecting blood flow such as Reynaud's syndrome, impotence, premature ejaculation, female anoryasmia, clitoral erectile insufficiency, vaginal engorgement, dyspareunia and vaginismus. Additionally retigabine may be useful for achieving reversible cardiac arrest and restoring coronary blood flow. Retigabine may also be useful for the treatment of neurodegeneration. Other disorders that may be treated by retigabine include intermittent claudication, pollakiuria, nocturia, hyperreflexia, enuresis, alopecia, dysmenorrheal, benign prostatic hyperplasia, premature labour, disorders associated with diabetes, such as retinopathy, neuropathy, nephropathy, peripheral circulation disorder and skin ulceration. Additionally retigabine may also be useful for treating behavioural disorders such as nicotine addiction withdrawal, mania, bipolar disease and anxiety disorders. These disorders are herein after referred to as the Disorders of the Invention.

Retigabine has been found to exhibit marked pH dependent solubility, i.e. it is more soluble in the acidic conditions of the stomach (around pH 2) than in the near neutral conditions of the lower intestine (around pH 7).

It is an object of the present invention to provide an oral dosage form comprising retigabine or a pharmaceutically acceptable salt or solvate thereof, which provides a mean (over a patient group) flattened plasma profile for an extended period of, time for example 4 to 24 hours, for example 4 to 15 hours, 4 to 12 after administration. Such a dosage form is considered to be suitable for twice daily or even once daily administration. Such a dosage form is also indicated for administration in both fasted and fed states, with substantially no clinically relevant food effect, i.e. no dose dumping under fed conditions. Currently in clinical trials retigabine immediate release (IR) tablets are administered three times a day. Additionally the oral dosage forms of the present invention may provide less interpatient variability in the pharmacokinetics than previous modified release formulations of retigabine.

Accordingly the present invention provides an oral dosage form comprising:

(i) an erodable core, which core comprises a first modified release composition comprising retigabine or a pharmaceutically acceptable salt or solvate thereof, and a pharmaceutically acceptable carrier therefore; and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

A further aspect of the invention is an oral dosage form wherein

(i) the erodable core comprising:
(a) a first modified release composition and
(b) a second composition;
   each composition comprising retigabine or a pharmaceutically acceptable salt or solvate thereof, and a pharmaceutically acceptable carrier therefore, wherein the first and second compositions are arranged to release drug at differing release rates on administration such that the rate of release of the drug from the dosage form is substantially independent of pH and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

As used herein, the term “modified release” means a composition which has been designed to produce a desired pharmacokinetic profile by choice of formulation. For example, the term “modified release” shall comprise delayed, pulsed and extended (sustained) release either alone or in any combination.

In one aspect the modified release composition provides extended release of retigabine or a pharmaceutically acceptable salt or solvate thereof.

Suitably, the release rate of the drug from the second composition (when present) is substantially greater than from the first composition. It is envisaged that the second composition is an immediate release composition.

Suitably said erodable coating is an enteric coating layer, most preferably a non-permeable enteric coating layer.

Suitably, the second composition (when present) is formulated so that it provides immediate release of retigabine or a pharmaceutically acceptable salt or solvate thereof, on contact with aqueous media. Suitably, the first composition is formulated so that it provides modified release of retigabine or a pharmaceutically acceptable salt or solvate thereof, on contact with aqueous media.

Suitably the first composition is arranged so that in use it releases at least 90% of the retigabine or a pharmaceutically acceptable salt or solvate thereof in the intestines.

Suitably the first composition is arranged so that in use it releases at least 95% of retigabine or a pharmaceutically acceptable salt or solvate thereof in the intestines.

Suitably the second composition is arranged so that in use it releases at least 50% of the retigabine or a pharmaceutically acceptable salt or solvate thereof, in the stomach.

Suitably the second composition is arranged so that in use it releases at least 60% of the retigabine or a pharmaceutically acceptable salt or solvate thereof, in the stomach.

Suitably the second composition is arranged so that in use it releases at least 75% of the retigabine or a pharmaceutically acceptable salt or solvate thereof, in the stomach.

Suitably, the dosage form is a tablet form.

During human trials of an embodiment of the oral dosage form of the invention we have found that, release of the drug is such that the mean maximum plasma level concentration (“Cₘₐₓ”) value of the drug is maintained substantially independent of food during use, i.e. the observed Cₘₐₓ value is similar in both fasted and fed states during use. Accordingly, in one aspect the oral dosage form is arranged to release retigabine or a pharmaceutically acceptable salt or solvate thereof, such that the mean maximum plasma level concentration (“Cₘₐₓ”) value of the drug is maintained substantially independent of food during use, i.e. no dose dumping occurred in the fed states during use.

In addition it has also been found that the oral dosage form releases the drug such that the mean area under the plasma concentration versus time curve over the dosing interval at steady state (“AUC”) observed on administration is maintained substantially independent of food during use, i.e. the observed AUC is similar in both fasted and fed states during use. Accordingly in one aspect the oral dosage form is arranged to release retigabine or a pharmaceutically acceptable salt or solvate thereof, such that the mean area under the plasma concentration versus time curve over the dosing interval at steady state (“AUC”) is maintained substantially independent of food during use, i.e. the observed AUC is similar in both fasted and fed states during use.

Thus, in a further aspect in operation the oral dosage form releases retigabine or a pharmaceutically acceptable salt or solvate thereof, so that both the Cₘₐₓ value and AUC observed on administration are maintained substantially independent of food during use, i.e. no dose dumping occurs in the fed states during use.

A further aspect of the invention is an oral dosage form which after administration may release the drug such that the mean area under the plasma concentration versus time curve over the dosing interval at steady state (“AUC”) is 80-125% of an equivalent dose of drug administered as IR tablets.

The compositions can be formed in any shape or mutual conformation providing the required objective of the invention is met.

The above reference to the core being erodable includes the situation where the core disintegrates partially or wholly, or dissolves, or becomes porous, on contact with the relevant environmental fluid so as to allow the fluid to contact the active agent. Suitably, the core disintegrates partially. Suitably, the core disintegrates wholly. Suitably, the core dissolves. Suitably, the core becomes porous.

The preferred embodiment of this invention provides that erosion of the coating is pH dependent.

Most suitably, although retigabine or a pharmaceutically acceptable salt or solvate thereof is more soluble in the stomach than the intestines, the product is formulated so as to release drug at suitable release rates in both the stomach and the intestines to generate a substantially flat pharmacokinetic profile relative to the immediate release formulation, i.e. the product is formulated to compensate for the pH dependency of retigabine.

The above reference to the coating being erodable includes the situation where the coating disintegrates partially or wholly, or dissolves, or becomes porous, on contact with an environmental fluid so as to allow the fluid to contact the core. Suitably, the coating disintegrates partially. suitably, the coating disintegrates wholly. Suitably, the coating dissolves. Suitably, the coating becomes porous. Preferably, the erodable coating is an enteric coating, i.e. it has a defined, pre-determined pH threshold at which it dissolves. Preferably, the coating erodes a pH greater than 4.5. More preferably, the coating erodes in the pH range from 4.5 to 8. Most preferably, the coating erodes in the pH range 5 to 7. Preferably, the enteric coating is non-permeable.

Materials and their blends suitable for use as an erodable coating material in this invention include various polymethacrylate polymers, co-processed polyvinylacetate phthalate, cellulose acetate trimelitate, cellulose acetate phthalate, shellac, hydroxyropylmethylcellulose phthalate polymers and their copolymers and hypromellose acetate succinate. Suitably, the coating material is selected from cellulose acetate trimelitate (CAT), polyvinyl acetate phthalate, hydroxypropylmethylcellulose phthalate 50, hydroxpropymethylcellulose phthalate 55, Acryl-eze™, Aquateric™, cellulose acetate phthalate, Eudragit™ L30 D55, Eudragit™ L, Eudragit™ FS, Eudragit™ S and shellac. Most preferably, the coating material is Eudragit™ L30 D55.

When necessary, the erodable coating may be modified by addition of plasticisers or anti-tact agents. Suitable materials for this purpose include waxy materials such as glycerides, for example glyceryl monostearate, or mono-/di-glycerides.

Typical sizes for the opening(s), when circular, to be formed in the coating are in the range 0.9 mm-6 mm of diameter, such as 1, 2, 3, 4 or 5 mms in diameter, depending on the overall size of the tablet and the desired rate of release. Additionally the opening may be 2.5 or 4.5 mm in diameter. In one aspect of the invention the openings are circular with diameters of 2 or 4 mms. In another aspect of the invention the openings are circular with the diameters of 3, 4 or 5 mm. In another aspect of the invention the openings are circular with diameters of 2.5, 3, 4, 4.5 or 5 mm. The opening(s) may have any convenient geometrical shape, but a rounded shape, e.g. substantially circular or elliptical is generally preferred. More elaborate shapes, such as text characters or graphics, may also be formed, provided that the release rate can be made uniform in individual dosage forms. Typical sizes of non-circular openings are equivalent in area to the above mentioned sizes for circular openings, thus in the range of from about 0.6 to about 30 mm².

For the purposes of the present invention, the term “opening” is synonymous with hole, aperture, orifice, passageway, outlet etc. The opening(s) may be formed by methods disclosed in U.S. Pat. No. 5,004,614. Typically opening(s) may be formed by drilling, for example using mechanical drill bits or laser beams, or by punches that remove the cut area. The formation of the opening(s) may be default remove a small portion of the exposed core. It is also possible to purposely form a cavity below the aperture as a release rate controlling device, the cavity exposing a greater initial surface area of core than a flat surface. Suitably, the opening(s) extend through the entire erodable coating such that there is immediate exposure of the core to the environmental fluid when the device is placed in the desired environment of use.

Also it is possible to form the opening(s) in situ when the dosage form is administered by forming a coating containing pore-forming agents i.e. material that will dissolve in the stomach to create pores in the coating. Typically the pore forming agent is erodable in the pH range from 1 to 3.

In a preferred embodiment the opening(s) are mechanically drilled.

In U.S. Pat. No. 5,004,614, the opening(s) preferably comprise about 10-60% of the total face area of the tablet i.e. the upper and lower surfaces of a biconvex tablet. In the present invention, the opening(s) may comprise 0.18 to 20%, such as 1 to 20% of the total face area.

Alternatively, it may be useful to characterise the rate controlling effect of the opening(s) by reference to the area of the opening(s) relative to the total surface area of the coated tablet. Additionally, especially in cases where the core erodes by undercutting of the edges of the opening(s), the rate controlling effect may be related to the total circumference of the opening(s).

One finding is that two openings, for example one of each primary surface of a biconvex tablet, release an active agent from the core at a rate marginally greater than that of a single opening of the same overall area. It is also indicated that the variability of the release rate from the two openings is less than the variability of release rate from the corresponding single opening. Accordingly, in one embodiment of the invention, the coating of the core is provided with two or more openings. More preferably, the erodable coating surrounding the core is provided with two or more openings extending substantially completely through said coating but not penetrating said core and communicating from the environment of use to said core.

Where more than one opening is provided, the openings may be located on the same surface of the oral dosage form, or on different surfaces. Suitably, the oral dosage form has two openings, for example one on each of opposing surfaces. Suitably, the oral dosage form is a tablet having two opposed primary surfaces, each surface having one opening through the coating, preferably substantially completely through the coating. The core is suitably arranged so that one opening provides access to the first composition and the another opening provides access to the second composition. When the core does not include a second composition, the oral dosage form may still have two openings. It will be understood that both openings provide access to the first composition.

As a protection for the core material, to prevent contamination via the opening(s) before dosing, it may desirable to provide a conventional seal coating to either the core, or to the dosage form after formation of the opening(s). The seal coat may be a sub-coat or over-coat to the erodable coating.

Additional it may be desirable to provide a colour coating to either the core, or to the dosage form after formation of the opening(s). The seal coat may be a sub-coat or over-coat to the erodable coating.

A further aspect of the present invention is a process for preparing an oral dosage form comprising,

(i) an erodable core, which core comprises a first modified release composition comprising retigabine or a pharmaceutically acceptable salt or solvate thereof, and a pharmaceutically acceptable carrier therefore; and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions which process comprises at least the steps of

(i) formulating retigabine or a pharmaceutically acceptable salt or solvate thereof into a core;

(ii) coating the said core with an pH dependent erodable coating; and

(iii) creating one or more openings in the coating, said openings extending substantially completely through said coating but not penetrating said core and communicating from the environment of use to said core.

According to yet a further aspect of the present invention, there is provided a process for preparing an oral dosage form which dosage form comprises

(a) a first modified release composition and

(b) a second composition, each composition comprising retigabine or a pharmaceutically acceptable salt or solvate thereof (“the drug”) and a pharmaceutically acceptable carrier therefor, wherein the first and second compositions are arranged to release drug at differing release rates on administration such that the rate of release of the drug from the dosage form is substantially independent of pH;

which process comprises at least the steps of sequentially or simultaneously:

(i) formulating the drug into the first composition; and

(ii) formulating the drug into the second composition;

(iii) coating the said core with an pH dependent erodable coating; and

(iv) creating one or more openings in the coating, said openings extending substantially completely through said coating but not penetrating said core and communicating from the environment of use to said core.

The first and second compositions may be prepared by compressing suitable ingredients in conventional manner to form a compacted mass in multiple layers, which comprises the core of the dosage form (also referred to herein as “tablet core”). The tablet core may be prepared using conventional table excipients and formulation compression methods. Thus, the core typically comprises the active agent or agents along with excipients that impart satisfactory processing and compression characteristics such as diluents, binders and lubricants. Additional excipients that may form part of the core of the device include disintegrants, flavourants, colorants, release modifying agents and/or solubilising agents such as surfactants, pH modifiers and complexation vehicles. Typically, the active agent and excipients are thoroughly mixed prior to compression into a solid core. The core of the device may be formed by wet granulation methods, dry granulation methods or by direct compression. The core may be produced according to any desired pre-selected shape such as bi-convex, hemi-spherical, near hemi-spherical, round, oval, generally ellipsoidal, oblong, generally cylindrical or polyhedral, e.g. a triangular prism shape. The term “near hemi-spherical” is intended to be construed in the manner described in U.S. Pat. No. 5,004,614. Suitably, the core is formulated into a bi-convex shape, e.g. having two domed opposite surfaces.

Where the core comprises two compositions, a first composition and a second composition, one of these compositions is very lightly compressed, then the ingredients for the other composition are added and the two compositions are compressed together.

Suitable materials for the first composition are a rate controlling polymer or matrix forming polymer for example high molecular weight hypromellose (HPMC) 2910 (also known as E) or 2208 (also known as K), methylcellulose, polyethylene oxide, hydroxypropyl cellulose, xanthan gum, guar gum, locust bean gum, methyl cellulose, Eudragit N M, Eudragit N E, Kollidon S R, galactomannans, dextran, ethylcellulose, carbomer, carbopol, polycarbonphil, sodium carboxymethylcellulose, hydroxyethylcellulose, hydroxyethylmethylcellulose, shellac, zein, cellulose acetate or combinations thereof.

In one aspect of the invention high or low molecular weight hypromellose 2910 (also know as E) or 2208 (also known as K) or a mixture thereof may be used. Suitably the rate controlling polymer for the first composition is high or low molecular weight hypromellose 2208 (also known as K) or a mixture thereof. If only one composition is present in the core, suitably the rate controlling polymer for the first composition may be high or low molecular weight hypromellose 2910 (also known as E) or a mixture thereof. In a further aspect of the invention a combination of at least two polymers is used. In one aspect of the invention a combination of high and low molecular weight hypromellose polymers are used i.e. two polymers hypromellose K100LV and hypromellose K3LV. In one aspect of the invention the polymers are used in a ration between 75:25 to 15:85 of high molecular weight polymer:low molecular weight polymer.

In one aspect of the invention the rate controlling polymer or matrix forming polymer comprises 8-40%, preferably 10-30% by tablet weight.

Additionally a lubricant may be added for example magnesium stearate, sodium stearyl fumerate, talc or stearic acid.

In one aspect of the invention the lubricant comprises 0.4 to 1.5%, preferably 0.5 to 0.8% by tablet weight.

Additionally a filler may be added for example microcrystalline cellulose (AVICEL™), mannitol, or lactose.

In one aspect of the invention the filler comprises 0 to 77%, preferably 10 to 77% for example 10 to 30% by tablet weight.

Additionally a surfactant may be added for example sodium lauryl sulphate. In the present invention if a surfactant is used it is present in less than 1% preferably less than 0.5% of total tablet weight, 0 to 0.5% of total tablet weight.

Suitable materials for the second composition, include saccharoses, for example lactose and maltose, mannitol, xylitol, calcium lactate, calcium silicate, dicalcium phosphate, trehalose or microcrystalline cellulose for example Avicel™. Additionally disintergrants or superdisintergrants such as croscarmellose sodium or sodium starch glycolate, surfactants such as sodium lauryl sulphate could be added. Most suitably, the second composition is predominantly mannitol. More suitably, the second composition comprises as excipients, mannitol and magnesium stearate.

In one aspect of the invention the excipents in the second composition comprises 0 to 80%, preferably 55% by tablet weight.

In one aspect of the invention the disintergrants in the second composition comprises 0 to 5%, preferably 1% by tablet weight.

In a further aspect the retigabine may be wet granulated with other ingredients, selected from for example mannitol, Avicel™ or HPMC to prepare granules which are then blended with other ingredients to form the first and second compositions which are then compressed as discussed above. The wet granulation process can be performed in a fluid bed processor, where the dry powder is fluidized by incoming air through the bottom of the equipment and the binder solution is sprayed into the fluidized powder. The wet powder is dried to the appropriate moisture level. Alternatively, the wet granulation process can be performed in a high shear mixer or an extruder or similar unit setup for continuous wet granulation. In one aspect of the invention the same granule can be used in both composition or alternatively separate granules could be prepared for each composition.

In a further aspect of the invention the retigabine may be micronized. Formulations containing micronized retigabine may allow administration of formulations containing lower drug quantities. They may provide more consistent pharmacokinetic profiles, and may allow reduction in dosing regimen.

Fluid energy milling or micronisation is a frequently used process for size reducing pharmaceutical materials. The parent Active Pharmaceutical Ingredient (API) is fed into the milling chamber at a feed rate which is defined in the batch record and set up at the start of each batch. This product feed rate is monitored at intervals throughout the run. The injection gas creates a reduced pressure zone at the venturi, and the powder is pulled into the milling chamber.

The mill has a number of nozzles which are evenly spaced along the interior wall of the milling chamber to create the necessary momentum for collisions to reduce the size of the input API. The milling chamber acts as a particle classifier by keeping larger particles inside the chamber through inertia and allowing smaller particles to escape with the gas into the collection bag through the internal classifier.

Size reduction is achieved primarily through particle-particle collisions.

In one embodiment when the core does not include the second composition, the first modified release composition comprises micronized retigabine.

The core may be coated with a suitable pH dependent erodable material by any pharmaceutically acceptable coating method. Examples include coating methods disclosed in U.S. Pat. No. 5,004,614 and film coating, sugar coating, spray coating, dip coating, compression coating, electrostatic coating. Typical methods include spraying the coating onto the table core in a rotating pan coater or in a fluidised bed coater until the desired coating thickness is achieved. Suitably the coating is provided to add about 4 to 8 mg/cm² or 5-7 mg/cm² of dry polymer around the tablet surface area. Typically this results in an increase in weight (relative to the core) of from for example 3-10%, or 5-10% by weight. Suitably, the coating has a thickness in the range 0.04 to 0.5 mm.

As indicated above, the oral dosage form of the present invention is considered to be suitable for twice or once daily administration and during use is indicated to provide a therapeutic effect over an extended period of time, such as up to 24 hours, for example, up to 12, 14, 16, 18, 20 and 24 hours, per unit dose.

In a further aspect of the invention the oral dosage form provides extended release retigabine or a pharmaceutically acceptable salt or solvate thereof, for example providing in vivo release of the active agent over a time period of up to 48 hours for example up to 26 hours; suitably up to 4 to 24 hours; preferably up to 4 to 15 hours and for example up to 4 to 12 hours. In a further aspect of the invention the oral dosage form provides extended release retigabine or a pharmaceutically acceptable salt or solvate thereof, for example providing in vivo release of the active agent over a time period of at lease 12 hours and up to 48 hours for example at least 12 hours and up to 24 hours.

In yet a further aspect of the invention the oral dosage form provides pulsed release retigabine or a pharmaceutically acceptable salt or solvate thereof, for example providing up to 4, for example 2, pulses of active agent per 24 hours.

The quantity of retigabine or a pharmaceutically acceptable salt or solvate thereof to be used in accordance with the present invention is a matter to be determined based upon typical pharmaceutical considerations, e.g. known dosages for retigabine or a pharmaceutically acceptable salt or solvate thereof, and is not limited by the process of this invention.

In particular, where retigabine or a pharmaceutically acceptable salt or solvate thereof is used in accordance with the present invention, a suitable dosage range is up to 1500 mg, for example, 10 to 1500 mg, for example 20 to 800 mg, suitably 100 to 800 mg. Thus, suitable oral dosage forms of the invention comprise 40, 75, 80, 150, 160, 200, 300, 320, 400, 450, 480, 600 or 640 mg of retigabine or a pharmaceutically acceptable salt or solvate thereof.

The amount of retigabine or a pharmaceutically acceptable salt or solvate thereof present in the first composition and the second composition may be varied in accordance with the desired dissolution profile.

In one aspect of the invention the first composition comprises 1 to 4 for example 2 to 3 times as much retigabine as the second composition, suitably 1.5 to 2.5 for example 2 to 2.5 times.

For example, where the oral dosage form comprises 480 mg of retigabine or a pharmaceutically acceptable salt or solvate thereof, the table core suitably comprises a layer comprising about 340 mg of retigabine or a pharmaceutically acceptable salt or solvate thereof, and a layer comprising about 140 mg of retigabine or a pharmaceutically acceptable salt or solvate thereof.

By adjustment of the release rates of the first and second compositions, and adjusting the other variables mentioned above and the surface area of the exposed core, the release rates in the different environmental conditions can be harmonised to obtain comparable release rates under different body environments, and so achieve more constant dosing to a patient.

Dissolution rates may be assessed by in vitro testing in solutions of the appropriate pHs. For example, when comparing dissolution in the stomach and intestines, tests may be carried out initially at pH 1.3 with a transfer to pH 6.4 after 2 hours or 4 hours, as an assumed time for residence in the stomach before emptying into the intestines of a notional patient in respectively fasted and fed conditions.

A further aspect of the invention is an oral dosage form comprising:

(i) an erodable core, which core comprises a first modified release composition comprising retigabine or a pharmaceutically acceptable salt or solvate thereof, and a pharmaceutically acceptable carrier therefore; and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions which oral dosage form has dissolution profile wherein not less than 35% and not more than 65% retigabine is dissolved at 120 minutes and not less than 85% of retigabine is dissolved at 480 minutes when tested according to the dissolution method (2) wherein the test method employs USP Apparatus 2 equipment with 900 mL of media, a paddle speed of 100 rpm, a medium is 20 mM sodium phosphate with 1.0% w/v sodium dodecyl sulfate adjusted to pH 6.8.

A further aspect of the invention is an oral dosage form wherein

(i) the erodable core comprising;
a) a first modified release composition and
   b) a second composition;
      each composition comprising retigabine or a pharmaceutically acceptable salt or solvate thereof, and a pharmaceutically acceptable carrier therefore, wherein the first and second compositions are arranged to release drug at differing release rates on administration such that the rate of release of the drug from the dosage form is substantially independent of pH and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions which oral dosage form has dissolution profile wherein not less than 25% end not more than 65% retigabine is dissolved at 45 minutes and not less than 85% of retigabine is dissolved at 480 minutes when tested according to the dissolution method (2) wherein the test method employs USP Apparatus 2 equipment with 900 mL of media, a paddle speed of 100 rpm, a medium is 20 mM sodium phosphate with 1.0% w/v sodium dodecyl sulfate, adjusted to pH 6.8.

A further aspect of the invention is an oral dosage form wherein

(i) an erodable core comprising:
(a) a first modified release composition and
(b) a second composition;
   each composition comprising retigabine or a pharmaceutically acceptable salt or solvate thereof, and a pharmaceutically acceptable carrier therefore, wherein the first and second compositions are arranged to release of the drug from the dosage form is substantially independent of pH and

(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions which oral dosage form has dissolution profile wherein not more than 50% of retigabine is dissolved at 60 minutes and not less than 80% of retigabine is dissolved at 360 minutes in the dissolution test wherein the test method employs USP Apparatus 2 equipment with 900 mL of media and a paddle speed of 100 rpm, the medium is 20 mM sodium citrate with 2.0% w/v sodium dodecyl sulfate, adjusted to pH 6.4.

A further aspect of the invention is an extended release oral dosage form comprising a

(i) an erodable core, which core comprises
(a) 15% to 55% retigabine or a pharmaceutically acceptable salt or solvate thereof
(b) 8% to 40% of rate controlling polymer or matrix forming polymer
(c) 0 to 77% carrier; and

(ii) 3-10% by weight (relative to the core) of an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

A further aspect of the invention is an extended release oral dosage form wherein

(i) an erodable core comprising:
(a) a first modified release composition comprising
   (1) 15% to 55% retigabine or a pharmaceutically acceptable salt or solvate thereof
   (2) 8% to 40% of rate controlling polymer or matrix forming polymer
   (3) 0 to 77% carrier; and
(b) a second composition comprising
   (4) 20% to 60% retigabine or a pharmaceutically acceptable salt or solvate thereof
   (5) 0 to 80% carrier;
      wherein the first and second compositions are arranged to release drug at differing release rates on administration such that the rate of release of the drug from the dosage form is substantially independent of pH and

(ii) 3-10% by weight of an pH dependent erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

As mentioned above, retigabine or a pharmaceutically acceptable salt or solvate thereof when administered in an oral dosage form of this invention is indicated to be useful for the treatment and/or prophylaxis of the Disorders of the invention.

In one aspect of the invention, retigabine or a pharmaceutically acceptable salt or solvate thereof when administered in an oral dosage form of this invention is indicated to be useful for the treatment and/or prophylaxis of epilepsy.

In one embodiment the present invention provides a method for the treatment and/or prophylaxis of the Disorders of the Invention which method comprises administering an oral dosage form of this invention comprising retigabine or a pharmaceutically acceptable salt or solvate thereof, to a human or non-human mammal in need thereof.

In a preferred embodiment the present invention provides a method for the treatment and/or prophylaxis of epilepsy which method comprises administering an oral dosage form of this invention comprising retigabine or a pharmaceutically acceptable salt or solvate thereof, to a human or non-human mammal in need thereof.

In a further embodiment the present invention provides an oral dosage form of the invention comprising retigabine or a pharmaceutically acceptable salt or solvate thereof for use in the treatment and/or prophylaxis of the Disorders of the Invention.

In a further preferred embodiment the present invention provides an oral dosage form of the invention comprising retigabine or a pharmaceutically acceptable salt or solvate thereof for use in the treatment and/or prophylaxis of the epilepsy.

A further aspect to the invention is an oral dosage form according to the invention further comprising a second therapeutic agent. The second therapeutic agent may be selected from, but not limited to, carbamazepine (Tegretol™), valproate (Depakote™), tiagabine (Gabitril™), levetiracetam (Keppra™), gabapentin (Neurontin™), phenytoin (Dilantin™), lamotrigine (Lamictal™), clonazepam (Klonopin™), Clorazepate dipotassium (Tranxene™), acetazolamide (Diamox™), diazepam (Vallium™), ethosuximide (Zarontin™), felbamate (Felbatol™), fosphenytoin (Cerebyx™), lorazepam (Ativan™), oxcarbazepine (Trileptal™), phenobarbital, pregabalin (Lyrica™), primidone (Mysoline™), tiagabine hydrochloride (Gabratril™), topiramte (Topamax™), trimethadione (Tridone™), zonisamide (Zonegran™), lacosamide (Vimpat™), eslicarbazepine (Stedesa/Zebinix™), rufinamide (Banzel™), vigabatrin (Sabril™), brivaracetam (Rikelta™) and carisbamate (Comfyde). Appropriate doses of the second therapeutic agent will be readily appreciated by those skilled in the art.

As used herein, the term “pharmaceutically acceptable” embraces compounds, compositions and ingredients for both human and veterinary use. For example the term “pharmaceutically acceptable salt” embraces a veterinarily acceptable salt.

Suitable pharmaceutically acceptable solvates include hydrates.

As used herein, the term “Cₘₐₓ” shall mean the mean maximum plasma level concentration.

As used herein the term “AUC” shall mean the mean are under the plasma concentration versus time curve over the dosing interval at steady state.

No adverse toxicological effects are indicated in the above-mentioned treatments.

All publications, including but not limited to patents and patent applications, cited in this specification are herein incorporated by reference as if each individual publication were specifically and individually indicated to be incorporated by reference herein as though fully set forth.

These Examples are intended to be by way of illustration rather than limitation of the present invention.

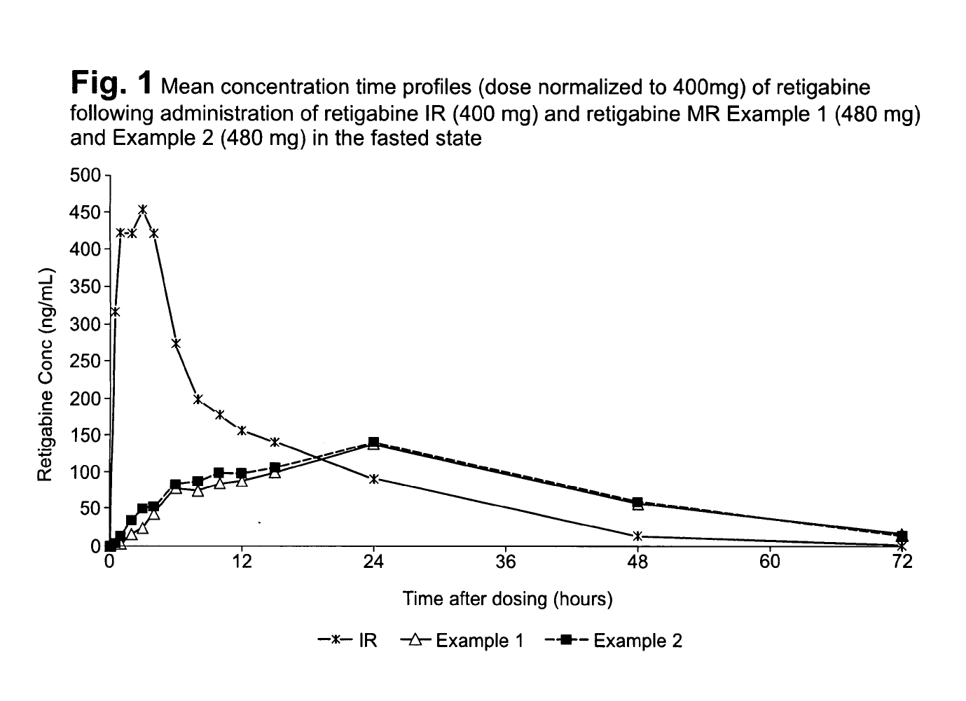

FIG. 1: Mean concentration time profiles (dose normalized to 400 mg) of retigabine following administration of retigabine IR (400 mg) and retigabine MR Example 1 (480 mg) and Example 2 (480 mg) in the fasted state

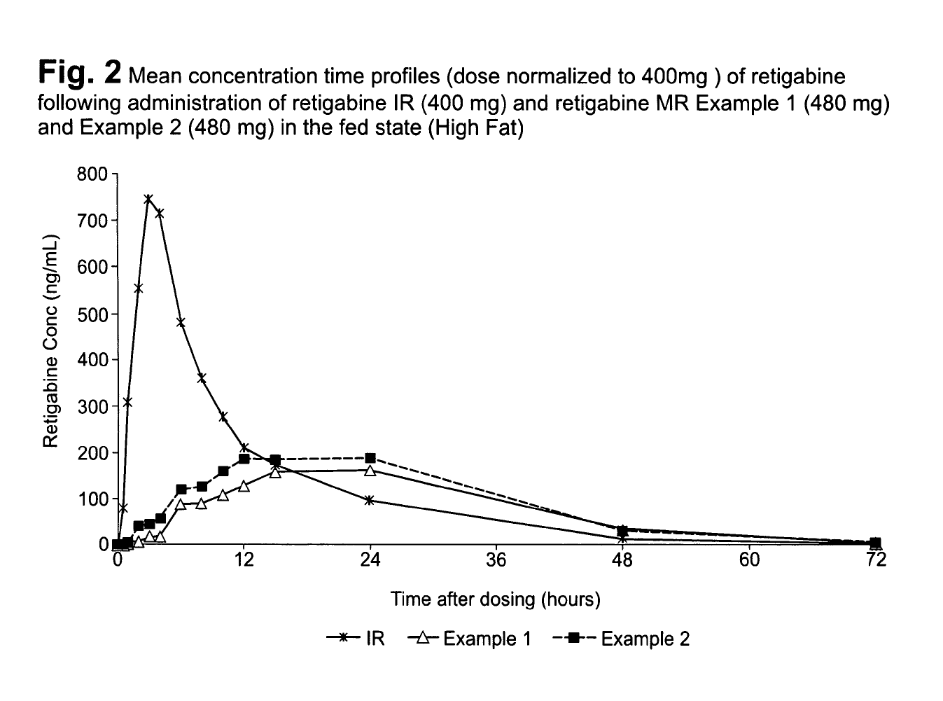

FIG. 2: Mean concentration time profiles (dose normalized to 400 mg) of retigabine following administration of retigabine IR (400 mg) and retigabine MR Example 1 (480 mg) and Example 2 (480 mg) in the fed state (High Fat).

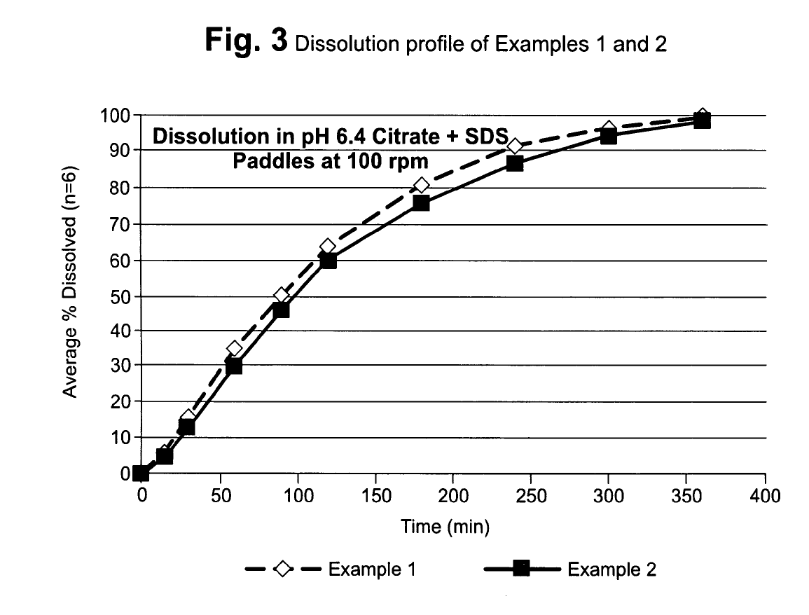

FIG. 3: Dissolution profile of Examples 1 and 2 tested with Dissolution Method (1).

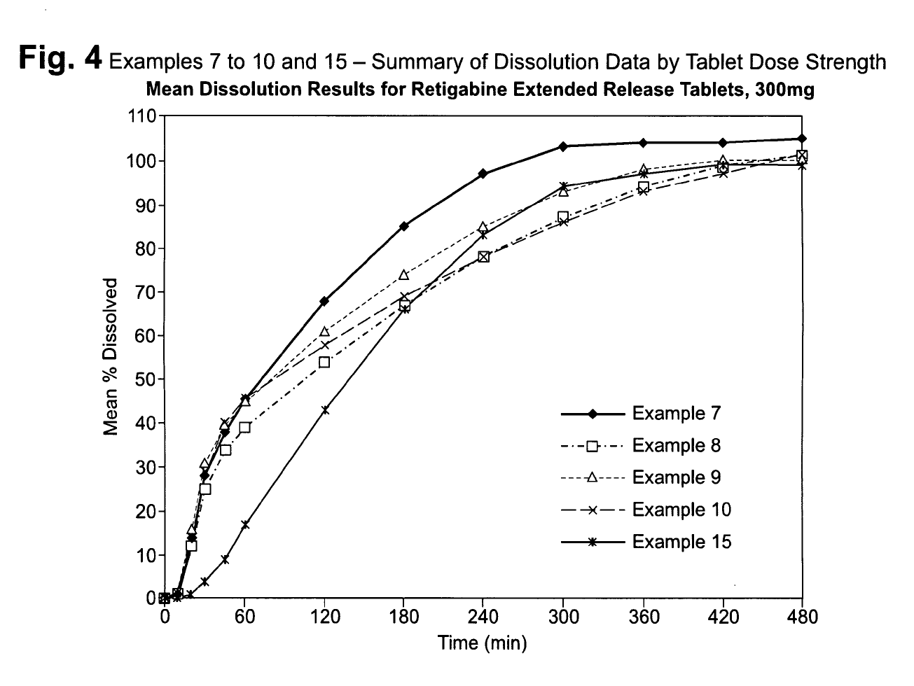

FIG. 4: Dissolution profile of Examples 7 to 10 and 15 tested with Dissolution Method (2).

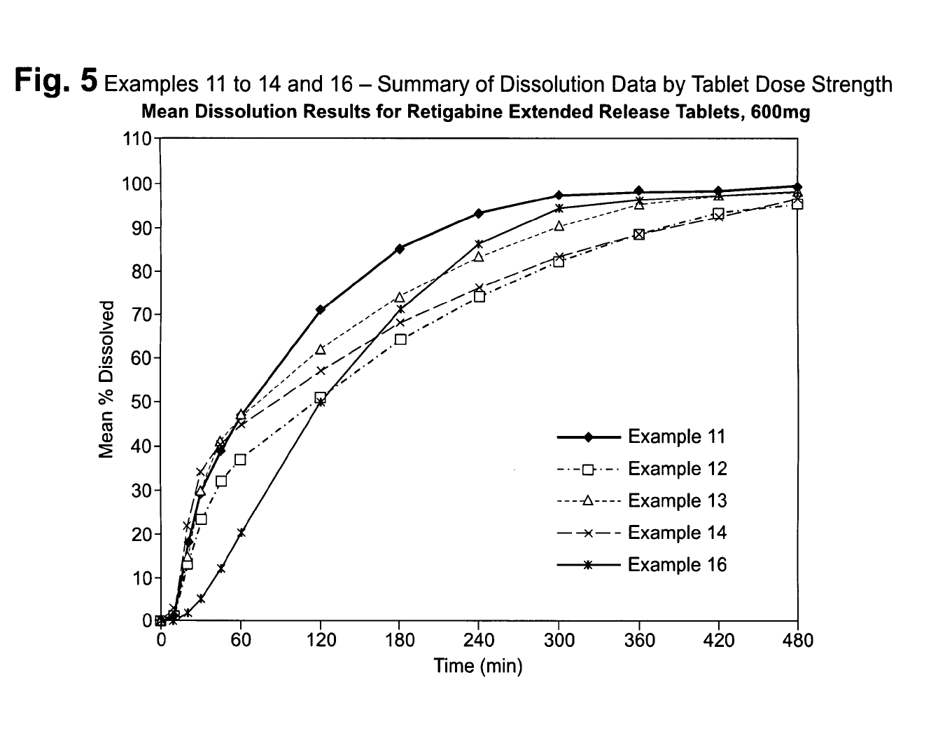

FIG. 5: Dissolution profile of Examples 11 to 14 and 16 tested with Dissolution Method (2)

A process for the preparation of retigabine.

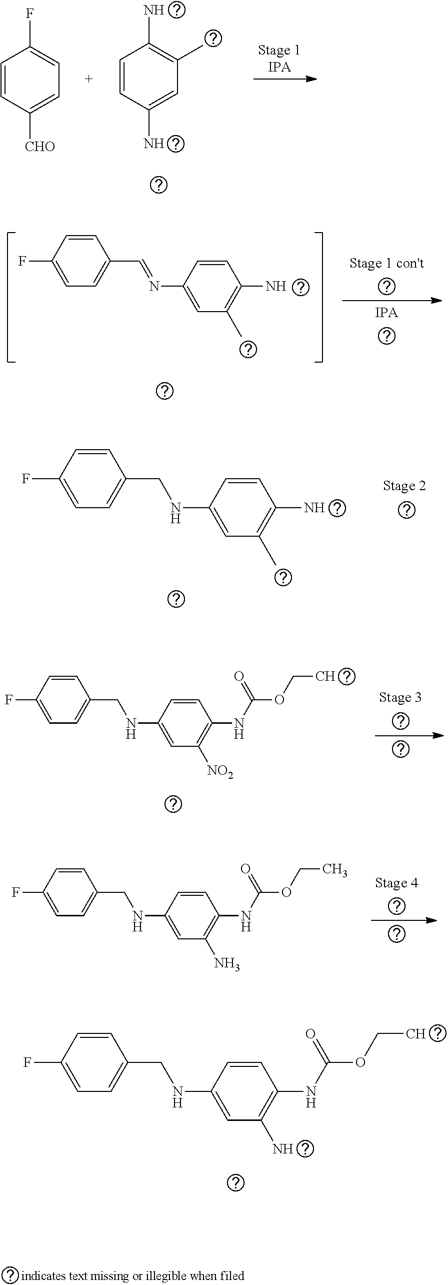

The following abbreviations are defined herein.

IPA—isopropanol or 2-propanol

IMS—Industrial methylated spirit

LOD—limit of detection.

### State 1-Preparation of 4-(4-fluorobenzylamino)-2-nitroaniline

4-Amino-2-nitroaniline (1.0 equiv.; 1.0 wt) was dissolved in IPA. The mixture was warmed to 75° C. and 4-fluorobenzaldehyde (1.05 equiv.; 0.285 wt) was added. When formation of imine was complete, a solution of NaBH₄ in 0.1% NaOH was added. After complete reduction of the imine at 75° C., water was added to the hot mixture. The mixture was cooled and acetone (0.2 vol) was added. The mixture was cooled to 15° C. and held for at least 30 minutes. Dark brown crystalline solid was collected, washed with water, and dried under vacuum at 50-55° C.

Percent yield range observed: 79-85%

### State 2-Preparation of 2-ethoxycarbonylamino-5-(4-fluorobenzylamino)-nitrobenzene

A vessel was charged with 4-(4-fluorobenzylamino)-2-nitroaniline (1.0 equiv.; 1.0 wt), NaOEt (2.0 equiv.; 0.52 wt), and diethylcarbonate (DEC) (7 vol). The heterogeneous mixture was stirred at 20-25° C. for 1.5 h or until complete by HPLC. Acetic acid (2.0 equiv) was charged and the mixture was heated to 40-50° C. H₂O and n-BuOH were added to the mixture and the layers were separated. The organic mixture was concentrated under vacuum and n-BuOH was added and distilled via constant volume distillation. The distillation was continued until the desired ratio was obtained. n-BuOH was added and the mixture was adjusted to 60-65° C. to dissolve all solids. The batch temperature was adjusted to 50° C. and seeded with 2-ethoxycarbonylamino-5-(4-fluorobenzylamino)-nitrobenzene. The suspension was stirred at 50° C. and then cooled to 0° C. The solid was filtered and washed with cold n-BuOH. The solid was dried under reduced pressure at 20-40° C.

Percent yield range observed: 80-88%

### State 3-Preparation of Retigabine

A pressure vessel was charged with 2-ethyoxycarbonylamino-5-(4-fluorobenzylamino)-nitrobenzene, 1 kg (1 wt), and the catalyst, 1% Pt +2% V/C, 50 g (0.05 wt). The vessel was pressure test with nitrogen to 6 barg. The reactor was charged with denatured ethanol, 10 L (10 vol), and the stir rate was set to >450 rpm. The vessel was pressure purged 3 times with nitrogen to 2 barg. The reaction mixture was heated to 50° C. under reactor control. Once an internal temperature of 50° C. was achieved, agitation was discontinued and the reactor purged three times with hydrogen to 2 barg. Following the third hydrogen purge and once the vessel reached 2 barg again, hydrogen flow control was initiated and the agitator activated. The reactor contents were aged for 2 hours. The reaction was heated to 70° C. and stirred for an addition 1 hour at 70° C. Once complete, the reaction mixture was filtered. The filtrate was transfer to a second 20 L vessel. The reactor was rinsed with denatured ethanol, 3 L (3 vol) and heated to >55° C. The rinse was filtered and the solution transferred to the second 20-L vessel. Once the batch temperature dropped below 30° C., a vacuum was established, 100 mbar (solution will boil at˜29° C.), and the solution concentrated to 7.5 L (7.5 vol). The solution was heated to 65° C. and aged until dissolution has occurred. The batch was cooled to 50° C. and seeded with retigabine (API) 5 g (0.005 wt) slurried in denatured ethanol, 20 mL (0.02 vol). After charging the seed, the solution was immediately cooled to 40° C. over 40 minutes, then aged for 60 min. The solution was cooled to 0° C. over 2 hours. The heterogeneous solution was stirred at 0° C. for 1 hour. The batch was milled, isolated and dried. The slurry was transferred to a filter and filtered. The wet cake was transferred to the vacuum oven and dried at 30-40° C. until the LOD indicated<0.5% wt. loss (120° C. for 15 minutes).

Percent yield range observed: 70-90%

### Preparation of Polymorph A

A reaction vessel was charged with denatured ethanol (7.0 volumes) and retigabine (1 wt) was added and the heated to 65-75° C. to dissolve, and stirred for 30 minutes. The solution was filtered to clarify with the temperature maintained above 60° C. throughout the filtration process to avoid precipitation of product. The reactor was rinsed and lines with 1 volume of denatured ethanol. After filtration, the filtered solution was reheated to 60-70° C. to ensure dissolution. The solution was cooled to 54-57° C. (55° C.) and temperature of the contents stabilized. The solution was cooled to 48-53° C., and upon reaching the desired temperature range, seeded with 0.5 wt % of Form A wet-milled seeds as a slurry in denatured ethanol (0.02 vol) at room temperature. The seed pot was washed with 0.02 vol denatured ethanol. The slurry was cooled to 30-40° C. (35° C.) and held for 60 minutes. The slurry was then cooled to −5° C. to 5° C. at up to 0.5° C./min.

The particle size was reduced using a wet mill on a reactor recirculation loop. When the target particle size was reached, the batch was heated to about 35° C. and then cooled to 0° C. and held for 30 min up to 24 hours. The slurry was charged to a filter dryer and settled for 30 min. The mother liquors were removed and the filter cake was washed with cold (0° C. ethanol wash) (2.0 volumes of denatured ethanol). Retigabine was isolated from the filter drier and was placed in appropriate containers.

Percent yield range observed: 85-89%

### EXAMPLE 1: 480 mg TABLET 2 mm APERTURE AND EXAMPLE 2: 480 mg TABLET 4 mm APERTURE

In the following example the granules for Layers 1 and 2 were prepared separately using standard wet granulation procedures. The granules were then blended with the remaining ingredients for each layer and compressed. The two layers were then compressed together, film coated using standard procedures to add the colour coat and then the enteric film coat was applied by spraying the coating onto the tablet core in a rotating pan coater. Apertures of 2 mm or 4 mm were mechanically drilled in the enteric coat producing tablets of Example 1 and 2 respectively.

TABLE 1

| | mg/tab |
|---|---|
| Layer 1, granulation | |
| Retigabine | 339.4 |
| Avicel PH101 | 77.4 |
| HPMC 603 | 16.3 |
| Total | 433.1 |
| Layer 2, granulation | |
| Retigabine | 145.4 |
| Mannitol, 160C | 27.3 |
| HPMC 603 | 9.1 |
| Total | 181.8 |
| Layer 1, Blend | |
| Layer 1 granules | 433.1 |
| HPMC, K100LV | 139.4 |
| Mannitol, 200SD | 71 |
| Mg Stearate | 6.1 |
| Total | 649.6 |
| Layer 2, Blend | |
| Layer 2 granules | 181.8 |
| Mannitol, 200SD | 57.1 |
| Mg Stearate | 1.5 |
| Total | 204.4 |
| Aqueous Film Coat | |
| Opadry, orange | 22 |
| Total | 912.0 |
| Enteric Film Coat | |
| Eudragit L30-D55 (dry basis) | 22.69 |
| Triethyl citrate | 2.38 |
| Mono-/di-glycerides | 0.65 |
| Polysorbate 80 | 0.28 |
| Totals | 938.0 |

Dissolution profiles for the dosage forms of Examples 1 and 2 are shown in FIG. 3 of the accompanying drawings.

The dissolution method used is as follows:

Dissolution is determined in accordance with USP General Chapter <711>. The procedure uses USP Apparatus 2 with a paddle speed of 100 rpm. The medium is 20 mM sodium citrate with 2.0% w/v sodium dodecyl sulphate, pH 6.4 (900 mL at 37C). The amount of dissolved retigabine is quantitated by UV spectroscopy using external standards.

### EXAMPLES 3, 4, 5 AND 6

Retigabine Common granules were prepared by wet granulation. The granules were manufactured by fluidizing the retigabine and microcrystalline cellulose powder and spraying the hypromeliose in solution onto the fluidized bed. After adding the appropriate amount of hypromellose, the wet granules were dried to an appropriate moisture level and milled to the desired particle size.

TABLE 2

| Description | % w/w |
|---|---|
| Retigabine | 78.4 |
| Microcrystalline Cellulose | 17.9 |
| Hypromellose 603 | 3.7 |
| Total | 100 |

These common granules were then used to prepare Layers 1 and 2 by mixing with the other ingredients and the layers were prepared by compression, aqueous film coated, then the enteric coat was applied. The apertures were mechanically drilled.

| | Ex. 3 | Ex. 4 | Ex. 5 | Ex. 6 |
|---|---|---|---|---|
| | 160 | 320 | 480 | 640 |
| Description | mg/tab | mg/tab | mg/tab | mg/tab |
| Layer 1, blend | | | | |
| Retigabine Common granule | 142.9 | 285.8 | 428.7 | 571.6 |
| Hypromellose, K100LV | 72.3 | 134.7 | 138.0 | 164.0 |
| Mannitol | 11.4 | 22.8 | 33.7 | 44.8 |
| Microcrystalline cellulose | 11.4 | 22.8 | 33.7 | 44.8 |
| Mg Stearate | 2.0 | 3.9 | 5.9 | 7.8 |
| Layer 1, Totals | 240.0 | 470.0 | 640.0 | 833.0 |
| Layer 2, Blend | | | | |
| Retigabine Common granule | 61.2 | 122.5 | 183.7 | 245.0 |
| Mannitol | 13.3 | 26.5 | 30.0 | 40.0 |
| Microcrystalline cellulose | 5.0 | 10.0 | 14.3 | 19.3 |
| Mg Stearate | 0.5 | 1.0 | 2.0 | 2.7 |
| Layer 2, Totals | 80.0 | 160.0 | 230.0 | 307.0 |
| Total Tablet weight | 320.0 | 630.0 | 870.0 | 1140.0 |
| Aqueous Film Coat | | | | |
| Opadry, orange | 13.0 | 19.0 | 23.0 | 27.0 |
| Totals | 333.0 | 649.0 | 893.0 | 1167.0 |
| Enteric Film Coat | | | | |
| Eudrgit L30 D55 (dry basis) | 14.84 | 22.69 | 27.93 | 33.16 |
| Triethyl citrate | 1.55 | 2.38 | 2.93 | 3.48 |
| Mono-/di-glycerides | 0.43 | 0.65 | 0.80 | 0.95 |
| Polysorbate 80 | 0.18 | 0.28 | 0.34 | 0.41 |
| totals | 350.0 | 675.0 | 925.0 | 1205.0 |
| Drilled Aperture Size (mm) | 2.5 mm | 3 mm | 4 mm | 5 mm |

Dissolution Method (1)

The test method employs USP Apparatus 2 equipment with 900 mL of media and a paddle speed of 100 rpm. The medium is 20 mM sodium citrate with 2.0% w/v sodium dodecyl sulfate, adjusted to pH 6.4. Sample aliquots are withdrawn at appropriate timepoints, clarified by filtration, and tested by UV spectrophotometry.

### A Study to Evaluate the Pharmacokinetics of Five Modified Release (MR) Formulations of Retigabine (480 mg) After Single Dose in Healthy Volunteers

Primary Objective

To investigate the bioavailability of five MR formulations (Examples 1 and 2 and three reference MR formulations) administered in the fed and fasted state as a single dose relative to administration of the immediate release (IR), in the fed and fasted state. To investigate the effect of food on the MR formulations administered as a single dose.

Study Design

This was an open-label, randomised single dose, cross-over phase I study conducted in healthy volunteers. Subjects were assigned to either a fasted dosing regimen (Group 1) or to a fed dosing regimen (Group 2 or Group 3) (high-fat meal) with a washout of 5-7 days between cross-over sessions. All meals were standardized. Each of the modified release formulations was administered in either the fasted or fed state.

Treatment Administration

Subjects in Part A, Group 1 received each of the MR formulations and the IR formulation in a randomised 6-way crossover fashion in the fasted state

TABLE 4
Dosing Regimens for Group 1(Fasted)
Group 1 (fasted)

400 mg IR (A)
480 mg MR Formulation 1—Example 2
480 mg MR Formulation 2—Example 1
480 mg MR Formulation 3—Reference formulation
480 mg MR Formulation 4—Reference Formulation
480 mg MR Formulation 5—Reference Formulation

Subjects in Group 2 received the IR formulation and the MR formulations in a randomised manner (4-way crossover) and subjects in Group 3 received IR formulation and 2 MR formulations in a randomised manner (3-way crossover) with a high fat meal (see Table 5)

TABLE 5
Dosing Regimens for Group 2 and Group 3(Fed)

| Part A, Group 2 (high fat meal) | Part A, Group 3 (high fat meal) |
|---|---|
| 400 mg IR (G) | 400 mg IR |
| 480 mg MR | 480 mg MR |
| Formulation 1—Example 2 | Formulation 4—Reference |
| | Formulation |
| 480 mg MR | 480 mg MR |
| Formulation 2—Example 1 | Formulation 5—Reference |
| | Formulation |
| 480 mg MR | |
| Formulation 3—Reference | |
| Formulation | |

Number and Nature of Subjects

Subjects were healthy adult male and female volunteers between 18 and 65 years of age (inclusive).

For Group 1, approximately 20 male subjects were to be enrolled such that approximately 16 subjects completed dosing and pharmacokinetic assessments.

For Group 2 and Group 3, approximately 12 male subjects were to be enrolled into each group such that approximately 10 subjects completed dosing and pharmacokinetic assessments

Pharmacokinetic Analysis

Pharmacokinetics (AUC and Cₘₐₓ) for immediate release and modified release formulation were the primary pharmacokinetic parameters. The secondary pharmacokinetic parameters were tₘₐₓ and t_{1/2} of immediate release and modified release formulations. Plasma samples for retigabine pharmacokinetic analysis were obtained prior to dosing and up to 72 hours post dose on each dosing occasion. Plasma concentrations for pharmacokinetic analysis of retigabine were determined by validated assay methodologies.

Mean retigabine concentration-time profiles following administration of retigabine IR and retigabine MR (Example 1 and Example 2) in the fasted state are shown in FIG. 1 and in the fed state (high fat meal) in FIG. 2.

For the MR formulations it was not possible to accurately determine the half-life, therefore AUC(0-t) was used as the primary endpoint. The PK results for this modified release formulation in the fasted and fed state are provided in Table 6.

TABLE 6
Summary of Dose Normalised PK parameters following
administration of IR tablets (400 mg) and Modified
Release tablets Example 1 and Example 2 (480 mg)
in Fasted State (geomean (CVb %)) (Group 1)

| | *Tmax | **Cmax | **AUC(0-t) |
|---|---|---|---|
| | (h) | (ng/mL) | (ng · h/mL) |
| IR (n = 18) | 2 (0.5-4) | 620 (28%) | 5720 (28%) |
| Example 1 (n = 18) | 24 (10-48) | 136 (40%) | 4670 (34%) |
| Example 2 (n = 19) | 24 (10-48) | 140 (29%) | 4640 (29%) |

*Median (range),
**Normalised to 400 mg.

TABLE 7
Summary of Dose Normalised PK parameters following
administration of IR Tablets (400 mg) and modified
release tablets Example 1 and Example 2 (480 mg)
in the Fed State (geomean (CVb %)) (Group 2)

| | *Tmax | **Cmax | **AUC(0-t) |
|---|---|---|---|
| | (h) | (ng/mL) | (ng · h/mL) |
| IR (n = 14) | 2.5 (0.5-4) | 840 (34%) | 6350 (24%) |
| Example 1 (n = 10) | 19.5 (6-24) | 189 (33%) | 4720 (25%) |
| Example 2 (n = 11) | 12 (6-24) | 236 (29%) | 5630 (21%) |

*Median (range),
**Normalised to 400 mg

In both the fasted and fed state administration of both Example 1 and Example 2 resulted in a reduction of Cmax to between 20 and 30% of that observed for IR. For both Example 1 and Example 2 in the fasted state, Tmax occurred between 10 and 48 hours post dose compared to between 0.5 and 4 hours for retigabine IR. For both Example 1 and Example 2 in the fed state, Tmax occurred between 6 and 24 hours post-dose compared to between 0.5 and 4 hours for retigabine.

In fasted state Formulation 3-Reference Example had a between subject variability for Cmax and AUC of 48% and 49%, respectively compared to 29% for Example 2.

### EXAMPLE 7 TO 14

The examples 7 through 14 below were prepared substantially as described in Example 3-6 using the retigabine common granules described in Table 2.

| | 300 mg Strength, Quantity by Formulation | | | | |
|---|---|---|---|---|---|
| | (mg/tablet) | | | | |
| Component | Example 7 | Example 8 | Example 9 | Example 10 | Function |
| First Composition: | | | | | |
| Modified Release Layer | | | | | |
| Retigabine | 210.0 | 210.0 | 180.0 | 180.0 | Active |
| Microcrystalline Cellulose | 47.9 | 47.9 | 41.1 | 41.1 | Filler |
| (Avicel PH101) | | | | | |
| Hypromellose 2910 | 10.1 | 10.1 | 8.6 | 8.6 | Binder |
| (Pharmacoat 603) | | | | | |
| Microcrystalline Cellulose | 47.0 | 47.0 | 42.1 | 42.1 | Filler |
| (Avicel PH200) | | | | | |
| Hypromellose 2208 (K3LV) | 105.0 | 70.0 | 60.0 | 36.0 | Modified |
| | | | | | Release |
| | | | | | Polymer |
| Hypromellose 2208 (K100LV) | 35.0 | 70.0 | 60.0 | 84.0 | Modified |
| | | | | | Release |
| | | | | | Polymer |
| Sodium Lauryl Sulfate | 2.5 | 2.5 | 2.1 | 2.1 | Surfactant |
| Magnesium Stearate | 2.5 | 2.5 | 2.1 | 2.1 | Lubricant |
| First Composition: | | | | | |
| Immediate Release Layer | | | | | |
| Retigabine | 90.0 | 90.0 | 120.0 | 120.0 | Active |
| Microcrystalline Cellulose | 20.5 | 20.5 | 27.4 | 27.4 | Filler |
| (Avicel PH101) | | | | | |
| Microcrystalline Cellulose | 43.0 | 43.0 | 50.4 | 50.4 | Filler |
| (Avicel PH102) | | | | | |
| Hypromellose 2910 | 4.3 | 4.3 | 5.8 | 5.8 | Binder |
| (Pharmacoat 603) | | | | | |
| Mannitol (Pearlitol 200SD) | 22.9 | 22.9 | 34.9 | 34.9 | Filler |
| Hypromellose, 2208 (K3LV) | 10.7 | 10.7 | 16.0 | 16.0 | Binder |
| Croscarmellose Sodium | 2.0 | 2.0 | 2.5 | 2.5 | Disintegrant |
| Iron Oxide Yellow | 0.1 | 0.1 | 0.1 | 0.1 | Colorant |
| Magnesium Stearate | 1.5 | 1.5 | 2.0 | 2.0 | Lubricant |
| Aqueous Sub-coat | | | | | |
| Opadry ® Brown | 19.0 | 19.0 | 19.0 | 19.0 | Coating |
| | | | | | Polymer |
| Enteric Film Coat | | | | | |
| Methacrylic Acid Copolymer | 22.69 | 22.69 | 22.69 | 22.69 | Modified |
| Dispersion¹ | | | | | Release |
| | | | | | Polymer |
| Triethyl Citrate | 2.37 | 2.37 | 2.37 | 2.37 | Plasticizer |
| Mono- and Di-Glycerides | 0.67 | 0.67 | 0.67 | 0.67 | Emulsifier |
| Polysorbate | 0.27 | 0.27 | 0.27 | 0.27 | Surfactant |
| Drilled Aperture Size | 3.0 mm | 3.0 mm | 3.0 mm | 3.0 mm | |

Note:
¹Dry basis.

Composition of Extended Release Tablets, 600 mg, Example 11, 12, 13 and 14

| | 600 mg Strength, Quantity by Formulation | | | | |
|---|---|---|---|---|---|
| | (mg/tablet) | | | | |
| Component | Example 11 | Example 12 | Example 13 | Example 14 | Function |
| First Composition: | | | | | |
| Modified Release Layer | | | | | |
| Retigabine | 420.0 | 420.0 | 360.0 | 360.0 | Active |
| Microcrystalline Cellulose | 95.8 | 95.8 | 82.1 | 82.1 | Filler |
| (Avicel PH101) | | | | | |
| Hypromellose 2910 | 20.2 | 20.2 | 17.3 | 17.3 | Binder |
| Pharmacoat 603) | | | | | |
| Microcrystalline Cellulose | 86.0 | 86.0 | 74.6 | 74.6 | Filler |
| (Avicel PH200) | | | | | |
| Hypromellose 2208 (K3LV) | 148.5 | 99.0 | 84.0 | 50.4 | Modified |
| | | | | | Release |
| | | | | | Polymer |
| Hypromellose 2208 (K100LV) | 49.5 | 99.0 | 84.0 | 117.6 | Modified |
| | | | | | Release |
| | | | | | Polymer |
| Sodium Lauryl Sulfate | 5.0 | 5.0 | 4.0 | 4.0 | Surfactant |
| Magnesium Stearate | 5.1 | 5.1 | 4.0 | 4.0 | Lubricant |
| Second Composition: | | | | | |
| Immediate Release Layer | | | | | |
| Retigabine | 180.0 | 180.0 | 240.0 | 240.0 | Active |
| Microcrystalline Cellulose | 41.1 | 41.1 | 54.7 | 54.7 | Filler |
| (Avicel PH101) | | | | | |
| Microcrystalline Cellulose | 70.3 | 70.3 | 85.0 | 85.0 | Filler |
| (Avicel PH102) | | | | | |
| Hypromellose 2910 | 8.6 | 8.6 | 11.5 | 11.5 | Binder |
| Pharmacoat 603) | | | | | |
| Mannitol (Pearlitol 200SD) | 49.8 | 49.8 | 59.8 | 59.8 | Filler |
| Hypromellose, 2208 (K3LV) | 23.0 | 23.0 | 30.0 | 30.0 | Binder |
| Croscarmellose Sodium | 4.0 | 4.0 | 5.0 | 5.0 | Disintegrant |
| Iron Oxide Yellow | 0.2 | 0.2 | 0.2 | 0.2 | Colorant |
| Magnesium Stearate | 3.0 | 3.0 | 3.8 | 3.8 | Lubricant |
| Aqueous Sub-coat | | | | | |
| Opadry ® Brown | 36.0 | 36.0 | 36.0 | 36.0 | Coating |
| | | | | | Polymer |
| Enteric Film Coat | | | | | |
| Methacrylic Acid Copolymer | 33.17 | 33.17 | 33.17 | 33.17 | Modified |
| Dispersion¹ | | | | | Release |
| | | | | | Polymer |
| Triethyl Citrate | 3.46 | 3.46 | 3.46 | 3.46 | Plasticizer |
| Mono- and Di-Glycerides | 0.97 | 0.97 | 0.97 | 0.97 | Emulsifier |
| Polysorbate | 0.40 | 0.40 | 0.40 | 0.40 | Surfactant |
| Drilled Aperture Size | 4.5 mm | 4.5 mm | 4.5 mm | 4.5 mm | |

Note:
¹Dry basis.

### Dissolution Method (2) for Examples 7 through 14

Examples 7 to 14 were tested using the following dissolution method.

The test method employs USP Apparatus 2 equipment with 900 mL of media and a paddle speed of 100 rpm. The medium is 20 mM sodium phosphate with 1.0% w/v sodium dodecyl sulfate, adjusted to pH 6.8. Sample aliquots are withdrawn at appropriate timepoints, clarified by filtration, and tested by UV spectrophotometry.

### EXAMPLES 15 AND 16

The retigabine drug substance used in Example 15 and 16 was micronized, as described in prior sections. Common granules were prepared by continuous wet granulation where components of the granule formulation were feed into the barrel of a continuous granulation followed by the continuous influx of the granulating solution. The wet granules were transferred to a fluid bed dryer and dried to the desired moisture level in a similar fashion as described for Examples 3 to 6. The granules were dry milled and mixed with the other ingredients followed by tablet compression, then aqueous film coated, then the enteric coat applied. The apertures were mechanically drilled.

Composition of Extended Release Tablets, Example 15 (300
mg dose strength) and Example 16 (600 mg dose strength)

| | Quantity by Strength | | |
|---|---|---|---|
| | (mg/tablet) | | |
| | Example 15 | Example 16 | |
| Component | (300 mg) | (600 mg) | Function |
| Micronized Retigabine | 300.0 | 600.0 | Active |
| Microcrystalline | 28.1 | 56.3 | Filler |
| Cellulose (Avicel PH101) | | | |
| Microcrystalline | 70.0 | 111.0 | Filler |
| Cellulose (Avicel PH102) | | | |
| Hypromellose 2208 | 42.5 | 65.0 | Modified |
| (K100LV) | | | Release |
| | | | Polymer |
| Hypromellose 2208 | 146.3 | 202.5 | Modified |
| (K3LV) | | | Release |
| | | | Polymer |
| Mannitol (Pearlitol 160C) | 28.1 | 56.3 | Filler |
| Magnesium Stearate | 5.0 | 9.0 | Lubricant |
| Aqueous Sub-coat | | | |
| Opadry ® Brown | 19.0 | 33.0 | Coating |
| | | | Polymer |
| Enteric Film Coat | | | |
| Methacrylic Acid | 22.69 | 33.17 | Modified |
| Copolymer Dispersion¹ | | | Release |
| | | | Polymer |
| Triethyl Citrate | 2.37 | 3.46 | Plasticizer |
| Mono- and Di-Glycerides | 0.67 | 0.97 | Emulsifier |
| Polysorbate | 0.27 | 0.40 | Surfactant |
| Drilled Aperture Size | 3.0 mm | 4.5 mm | |

### Dissolution Method (2) Examples 15 and 16

Examples 15 and 16 were tested using the following dissolution method. The test method employs USP Apparatus 2 equipment with 900 mL of media and a paddle speed of 100 rpm. The medium is 20 mM sodium phosphate with 1.0% w/v sodium dodecyl sulfate, adjusted to pH 6.8. Sample aliquots are withdrawn at appropriate timepoints, clarified by filtration, and tested by UV spectrophotometry.

### A Study to Evaluate Modified Release (MR) Formulations of Retigabine in Healthy Volunteers

For clinical evaluation of examples 7 through 16, the subjects will be up-titrated to a total daily dose of 600 mg. The purpose of the titration phase is to improve the tolerability of retigabine. Next, the relative bioavailability assessments will be conducted by switching the formulations on every fourth day of treatment. Each subject will be participating in six study periods, in five periods subjects will receive a 300 mg BID dosing of retigabine MR formulations (Examples 7 to 10 and 15) and in one period subjects will receive 200 mg TID dosing of retigabine IR. The allocation of the treatments will follow a crossover design with treatment sequences allocated according to a predefined randomisation schedule. After this crossover phase, the subjects will be randomised to one of the five MR formulations for the Food Effect phase. The effect of a high fat meal on the pharmacokinetics of retigabine following administration of each of the MR formulations (Examples 11 to 14 and 16) will be studied at a dose of 600 mg OD since any food effect would be anticipated to have a greater impact at the highest dose strength. This phase is to be conducted as a fixed sequence; the pharmacokinetics will be initially evaluated following administration of retigabine with a standard meal, followed by administration with a high-fat meal. The fixed sequence should reduce the impact of any dropouts due to tolerability issues should a food effect occur with one on the MR formulations. This phase of the study is important because it allows evaluation of the pharmacokinetics of retigabine following administration of the highest MR tablet strength (600 mg) and can be used to predict the systemic exposure to retigabine following administration at the upper end of the efficacious dose range of 1200 mg day (600 mg BID).

1. An oral dosage form comprising:
(i) an erodable core, which core comprises a first modified release composition comprising retigabine or a pharmaceutically acceptable salt or solvate thereof, and a pharmaceutically acceptable carrier therefore; and
(ii) an erodable coating around said core, which coating comprises one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

2. An oral dosage form as claimed in claim 1 wherein
(i) the erodable core comprises:
(a) a first modified release composition and
(b) a second composition;
each composition comprising retigabine or a pharmaceutically acceptable salt or solvate thereof, and a pharmaceutically acceptable carrier therefore, wherein the first and second compositions are arranged to release drug at differing release rates on administration such that the rate of release of the drug from the dosage form is substantially independent of pH.

3. An oral dosage form as claimed in claim 1 wherein the first composition comprises a rate controlling polymer or matrix forming polymer selected from, for example, high molecular weight hypromellose (HPMC) 2910 (also known as E) or 2208 (also known as K), methylcellulose, polyethylene oxide, hydroxypropyl cellulose, xanthan gum, guar gum, locust bean gum, Eudragit N M, Eudragit N E, Kollidon S R, galactomannans, dextran, ethylcellulose, carbomer, carbopol, polycarbophil, sodium carboxymethylcellulose, hydroxyethylcellulose, hydroxyethylmethylcellulose, shellac, zein, cellulose acetate or combinations thereof.

4. An oral dosage form as claimed in claim 3 wherein the rate controlling polymer or matrix forming polymer selected from, for example, high molecular weight hypromellose (HPMC) 2910 (also known as E).

5. An oral dosage form as claimed in claim 3 wherein the rate controlling polymer or matrix forming polymer selected from, for example, high molecular weight hypromellose (HPMC) 2208 (also known as K).

6. An oral dosage form as claimed in claim 1 wherein the coating erodes at pH greater than 4.5.

7. An oral dosage form as claimed in claim 1 wherein the opening(s) in the coat is/are in the range 0.9 to 6 mm in diameter.

8. An oral dosage form as claimed in claim 1 wherein the openings may comprise 0.18 to 20% of the total tablet face area.

9. An oral dosage form as claimed in claim 1 wherein the first composition comprises 2 to 3 times as much retigabine or pharmaceutically acceptable salt or solvate thereof as the second composition.

10. An oral dosage form as claimed in claim 1 comprising 10 to 1500 mg retigabine.

11-13. (canceled)

14. A method of treating a patient suffering from epilepsy comprising administering to the patient an oral dosage form as claimed in claim 1.

15. An oral dosage form as claimed in claim 1 wherein the retigabine is in micronized form.

16. An oral dosage form as claimed in claim 1 wherein the erodable core is coated with a seal coat.

17. An oral dosage form as claimed in claim 1 wherein the erodable coating around said core is over coated with a seal coat.

18. An oral dosage form as claimed in claim 1 for once daily administration.
